(19) United States
(12) Patent Application Publication
(10) Pub. No.: US 2016/0368941 A1
(43) Pub. Date: Dec. 22, 2016

(54) NOVEL METHODS OF ISOMERIZING CARBOHYDRATES

(71) Applicant: YALE UNIVERSITY, New Haven, CT (US)

(72) Inventors: Alanna Schepartz SHRADER, New Haven, CT (US); Aaron ZWICKER, New Haven, CT (US); Scott MILLER, Woodbridge, CT (US)

(21) Appl. No.: 15/121,422

(22) PCT Filed: Mar. 4, 2015
(86) PCT No.: PCT/US15/18567

§ 371 Date: Aug. 25, 2016

Related U.S. Application Data
(60) Provisional application No. 61/947,802, filed on Mar. 4, 2014.
(60) Provisional application No. 62/078,671, filed on Nov. 12, 2014.

(51) Int. Cl. C07H 3/02; C07H 1/00

(52) U.S. Cl. CPC C07H 3/02; C07H 1/00

(57) ABSTRACT

The invention provides a method of isomerizing a sugar into fructose using a calcium salt and an organic base. In certain embodiments, the sugar is glucose and/or mannose.

### CROSS-REFERENCE TO RELATED APPLICATIONS

The present application claims priority under 35 U.S.C. §119(e) to U.S. Provisional Patent Applications No. 61/947,712, filed Mar. 4, 2014, and No. 62/078,671, filed Nov. 12, 2014, all of which applications are incorporated herein by reference in their entireties.

### BACKGROUND OF THE INVENTION

Glucose, also known as D-glucose or dextrose, is a monosaccharide found in plants. This dietary monosaccharide is absorbed directly into the bloodstream during digestion, and cells use it as a source of energy. The open-chain form of glucose exists in equilibrium with several cyclic isomers, which may be pyranoses or furanoses. In aqueous solution, more than 99% of glucose molecules, at any given time, exist as pyranoses (α-D- or β-D-glucopyranoses). The open-chain form is limited to about 0.25%, and furanoses (β-D- or β-D-glucofuranoses) exist in negligible amounts.

Glucose is produced commercially via the enzymatic hydrolysis of starch, obtained from crops such as maize, rice, wheat, cassava, corn husk and sago. In the U.S., cornstarch (from maize) is used almost exclusively. In principle, cellulose could be hydrolyzed to glucose, but this process is not yet commercially practical.

Fructose is a monosaccharide found in many plants, where it is often covalently coupled to glucose to form the disaccharide sucrose. Natural sources of fructose include fruits, vegetables (including sugar cane) and honey. Commercially, fructose is often derived from sugar cane, sugar beets and maize. In solution, fructose exists as an equilibrium mixture of 70% fructopyranose and about 22% fructofuranose, as well as small amounts of other forms, including the acyclic structure.

Fructose, in the form of fruits and juices, is commonly added to foods and drinks for palatability and taste enhancement, and for browning of baked goods. Fructose is used commercially in foods and beverages because of its low cost and high relative sweetness; fructose is the sweetest of all naturally occurring carbohydrates, being about 1.73 times as sweet as sucrose.

Fructose is also found in the synthetically manufactured sweetener high-fructose corn syrup (HFCS), which is enzymatically produced from hydrolyzed corn starch. Through the enzymatic treatment, glucose molecules are converted into fructose molecules. As a result, HFCS comprises a mixture of glucose and fructose as monosaccharides. There are three types of HFCS—HFCS-42, HFCS-55, and HFCS-90—wherein the number indicates the percentage of fructose present in the syrup. HFCS-55 is used as sweetener in soft drinks, whereas HFCS-42 is used in many processed foods and baked goods.

The enzymatic process that converts corn starch into HFCS comprises the following enzymes: (a) α-amylase: converts cornstarch into glucose oligosaccharides; (b) glucoamylase: breaks the glucose oligosaccharides down even further to yield the simple sugar glucose; and (c) xylose isomerase (also known as glucose isomerase): converts glucose to a mixture of about 42% fructose, 50-52% glucose and about 6-8% of “other sugars.”

While inexpensive a-amylase and glucoamylase are added directly to the slurry and used only once, the more costly xylose isomerase is immobilized on a solid surface, over which the sugar mixture is passed. The isomerase is used repeatedly until it loses its catalytic activity. This 42% fructose glucose mixture is then subjected to a liquid chromatography step, where the fructose is enriched to about 90%. The 90% fructose is back-blended with 42% fructose to achieve a 55% fructose final product.

There are numerous drawbacks to this biocatalytic process. For the enzymatic reaction to take place, the glucose feedstock must be rigorously pre-purification. The buffer present in the reaction mixture must be removed. The enzymatic reaction is effective over only a narrow temperature range, and the immobilized xylose isomerase must be replaced as its turnout performance decreases over time.

Fructose readily dehydrates to give hydroxymethylfurfural (5-furfural or “HMF”), an useful chemical intermediate. The reaction generally involves treatment of fructose with acids, followed by liquid-liquid extraction of HMF into organic solvents, such as methyl isobutyl ketone. This process may soon become part of a low-cost, carbon-neutral system to produce plant-based replacements for petroleum and diesel. For example, HMF can be converted to 2,5-dimethylfuran, which is a high-energy content liquid biofuel. Oxidation of HMF gives 2,5-furan-dicarboxylic acid, which may be a replacement for terephthalic acid in the production of polyesters.

There is a need in the art to identify novel methods of converting glucose into fructose. Such methods should be cost-effective and avoid the use of easily inactivated enzymes. Such methods should further proceed in high yield and with minimal formation of unwanted decomposition products or side products. The present invention addresses and meets this need.

### BRIEF SUMMARY OF THE INVENTION

The invention includes a method of isomerizing a sugar into fructose.

In certain embodiments, the sugar is selected from the group consisting of glucose and mannose.

In certain embodiments, the method comprises contacting an aqueous solution of the sugar with a calcium salt and an organic base to form a system, wherein fructose is formed in the system. In other embodiments, the system is free of organic solvents. In yet other embodiments, the system comprises one or more water-soluble organic solvents.

In certain embodiments, the calcium salt comprises one or more anions selected from the group consisting of chloride, bromide, iodide, nitrate, bicarbonate, sulfate bisulfate, phosphate, hydrogen phosphate, dihydrogen phosphate, and aliphatic, cycloaliphatic, aromatic, araliphatic, heterocyclic, carboxylic and sulfonic classes of organic anions.

In certain embodiments, the organic base is aromatic or aliphatic. In other embodiments, the aliphatic organic base is at least one selected from the group consisting of trimethylamine, triethylamine, tri-n-propylamine, diisopropylethylamine, N-alkyl-pyrrolidine, N-alkyl-morpholine, and N-alkyl-piperidine.

In certain embodiments, the reaction is run from about 0° C. to about 100° C. In other embodiments, the reaction is run from about 20° C. to about 50° C.

In certain embodiments, the sugar, calcium salt and organic base are in solution. In other embodiments, at least one selected from the group consisting of the calcium salt and the organic base is not in solution. In yet other embodiments, at least one selected from the group consisting of the calcium salt and the organic base is a solid. In yet other embodiments, at least one selected from the group consisting of the calcium salt and the organic base is immobilized on a solid support or permeable membrane. In yet other embodiments, the calcium salt and the organic base are each independently immobilized on a solid support or permeable membrane.

In certain embodiments, the reaction is run for a period ranging from about 5 minutes to about 4 weeks. In other embodiments, the reaction is run for a period of about 3 days. In yet other embodiments, the reaction is run for a period of about 2 weeks. In yet other embodiments, the reaction is run under a pressure that is higher or lower than atmospheric pressure. In yet other embodiments, the sugar, calcium salt and organic base are contacted in the presence of microwave irradiation. In yet other embodiments, the reaction is quenched by a method comprising precipitation, filtration, decantation, crystallization, solvent extraction, resin extraction, resin neutralization or chromatography. In yet other embodiments, the fructose is isolated from the system using a method comprising precipitation, filtration, decantation, crystallization, solvent extraction, resin extraction or chromatography. In yet other embodiments, the system comprising fructose is further subjected to a process comprising precipitation, filtration, decantation, crystallization, solvent extraction, resin extraction or chromatography.

### BRIEF DESCRIPTION OF THE DRAWINGS

The following detailed description of specific embodiments of the invention will be better understood when read in conjunction with the appended drawings. For the purpose of illustrating the invention, shown in the drawings are specific embodiments. It should be understood, however, that the invention is not limited to the precise arrangements and instrumentalities of the embodiments shown in the drawings.

FIGS. 1A-1B illustrate the ¹H NMR spectrum (FIG. 1A) and ¹³C NMR spectrum (FIG. 1B) of a reaction aliquot product of Example 2.

FIGS. 2A-2B illustrate the ¹H NMR spectrum (FIG. 2A) and ¹³C NMR spectrum (FIG. 2B) of the isolated product of Example 2.

FIG. 3 illustrates the ¹³C NMR spectrum of commercially available fructose.

FIGS. 4A-4B illustrate the analysis of the reaction exemplified in Example 3. FIG. 4A illustrates HPLC analysis of reaction mixtures, and FIG. 4B illustrates ¹³C NMR analysis of reaction mixtures.

### DETAILED DESCRIPTION OF THE INVENTION

The invention relates in part to the unexpected discovery that glucose and/or mannose may be converted to fructose in good yield in an enzyme-free process. In one aspect, an aqueous solution of glucose and/or mannose is contacted with a calcium salt and an organic base, thereby generating fructose.

In certain embodiments, the reaction is performed at room temperature. In other embodiments, the reaction mixture is free of organic solvents. In yet other embodiments, the reaction mixture comprises one or more water-soluble organic solvents.

Without wishing to be limited by any theory, the method of the invention is advantageous over the current industrial method for glucose-fructose or mannose-fructose isomerization, because the method of the invention does not require the use of expensive and easily inactivated enzymes. In certain embodiments, the method of the invention utilizes very low cost reagents. In a non-limiting example, calcium nitrate costs about $ 150 per metric ton; and trimethylamine costs about $ 500 per metric ton. In certain embodiments, the method of the invention does not utilize enzymes, and thus the HFCS prepared using the method of the invention is not classified as a “non-natural material.” In other non-limiting embodiments, the product of the method is fructose locked in the furanose form by virtue of formation of a calcium-fructose complex and/or salt. In yet other embodiments, the conformation-locked furanose calcium complex can be used to generate HMF.

The invention should not be construed to be limited to glucose and/or mannose as the starting saccharide. In certain embodiments, the methods described herein may be used to isomerize any sugar with an unprotected hydroxyl group at its closed conformation anomeric center. The products of such reaction may be analyzed and isolated using the methods and techniques described herein.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, specific methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of specifically ±20%, specifically ±10%, specifically ±5%, specifically ±1%, or specifically ±0.1% from the specified value, as such variations are appropriate to perform the disclosed methods.

As used herein, the term “composition” refers to a mixture of at least one compound useful within the invention with an acceptable liquid (which may be a solvent) or solid. The composition may facilitate manipulation, transfer or use of the compound within the methods of the invention.

As used herein, the term “fructose” refers to the monosaccharide fructose, a solvate thereof, any cation complex thereof (such as a calcium complex thereof), or any complex thereof with a base (such as a cation complex thereof in the presence of a base, such as a calcium complex thereof in the presence of a base) . The term “fructose” refers to all possible cyclic or acyclic conformers of fructose.

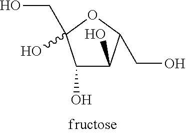

As used herein, the term “glucose” refers to the monosaccharide glucose or a solvate thereof. The term “glucose” refers to all possible cyclic or acyclic conformers of glucose.

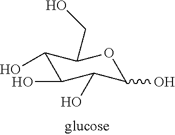 [SMILES: OCC1OC(O)C(O)C(O)C1O]

As used herein, the term “HMF” refers to 5-furfural or hydroxymethylfurfural, or a solvate thereof.

As used herein, the term “mannose” refers to the monosaccharide mannose or a solvate thereof. The term “mannose” refers to all possible cyclic or acyclic conformers of glucose.

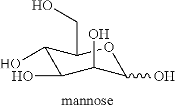 [SMILES: OCC1OC(O)C(O)C(O)C1O]

“Instructional material” as that term is used herein includes a publication, a recording, a diagram, or any other medium of expression that can be used to communicate the usefulness of the composition and/or method of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.1, 5.3, 5.5, and 6. This applies regardless of the breadth of the range.

### Methods

In one aspect, the invention comprises contacting an aqueous solution of glucose with a calcium salt and an organic base to form a system.

In certain embodiments, the system is free of organic solvents. As defined herein, the system is considered to be free of organic solvents is the only compound in the system is the organic base.

In certain embodiments, the aqueous solution of glucose comprises one or more water-soluble organic solvents. Non-limiting examples of water-soluble organic solvents contemplated within the invention comprise methanol, ethanol, n-propanol, isopropanol, dimethylsulfoxide, dimethylformamide, any mixtures thereof and the like.

In certain embodiments, the glucose is labelled with one or more isotopes, wherein one or more atoms is replaced by an atom having the same atomic number, but an atomic mass or mass number different from the atomic mass or mass number usually found in naturally occurring glucose. Examples of isotopes suitable for inclusion in the compounds described herein include and are not limited to ²H, ³H, ¹¹C, ¹³C, ¹⁴C, ¹⁵O, ¹⁷O, and ¹⁸O. In certain embodiments, isotopically labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C and ¹⁵O, is useful in Positron Emission Topography (PET) studies for examining substrate receptor occupancy. Isotopically labeled compounds are prepared by any suitable method or by processes using an appropriate isotopically labeled reagent in place of the non-labeled reagent otherwise employed.

In certain embodiments, the calcium salt comprises one or more anions selected from the group consisting of chloride, bromide, iodide, nitrate, bicarbonate, sulfate, bisulfate, phosphate, hydrogen phosphate, dihydrogen phosphate, and aliphatic, cycloaliphatic, aromatic, araliphatic, heterocyclic, carboxylic and sulfonic classes of organic anions. Non-limiting examples of organic anions include, but are not limited to, formate, acetate, propionate, succinate, glycolate, gluconate, lactate, malate, tartrate, citrate, ascorbate, glucuronate, maleate, malonate, saccharinate, fumarate, pyruvate, aspartate, glutamate, benzoate, anthranilate, 4-hydroxybenzoate, phenylacetate, mandelate, embonate (pamoate), methanesulfonate, ethanesulfonate, benzenesulfonate, pantothenate, trifluoromethanesulfonate, 2-hydroxyethanesulfonate, p-toluenesulfonate, sulfanilate, cyclohexylaminosulfonate, stearate, alginate, β-hydroxybutyrate, salicylate, galactarate and galacturonate acid.

In certain embodiments, the organic base is aromatic or aliphatic. Non-limiting examples of organic aliphatic bases contemplated within the invention are trimethylamine, triethylamine, tri-n-propylamine, diisopropylethylamine, N-alkyl-pyrrolidine, N-alkyl-morpholine, and N-alkyl-piperidine. Non-limiting examples of organic aromatic bases contemplated within the invention are pyridine and lutidines (such as 2,4-and 3,5-lutidines).

In certain embodiments, the glucose solution is contacted with the calcium salt and the organic base at a temperature ranging from about 0° C. to about 100° C. In other embodiments, the reaction is run at a temperature ranging from about 20° C. to about 50° C. In yet other embodiments, the reaction is run at about room temperature.

In certain embodiments, the glucose, calcium salt and organic base are in solution. In other embodiments, at least one selected from the group consisting of the calcium salt and the organic base is not in solution. In yet other embodiments, at least one selected from the group consisting of the calcium salt and organic base is a solid. In yet other embodiments, at least one selected from the group consisting of the calcium salt and the organic base is immobilized on a solid and/or permeable membrane. In yet other embodiments, the calcium salt and the organic base are each independently immobilized on a solid and/or permeable membrane.

In certain embodiments, the reaction is run for a period ranging from about 5 minutes to about 4 weeks. In other embodiments, the reaction is run for a period ranging about 1 hour to about 3 week, from about 2 hours to about 2 weeks, from about 3 hours to about 10 days, from about 4 hours to about 9 days, from about 5 hours to about 8 days, or from about 6 hours to about 6 days. In yet other embodiments, the reaction is run for a period of time of about 1 hour, 2 hours, 3 hours, 4 hours, 5 hours, 6 hours, 8 hours, 10 hours, 12 hours, 14 hours, 16 hours, 18 hours, 20 hours, 22 hours, 24 hours, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, or any fractions or multiple thereof.

In certain embodiments, the reaction is run under a pressure that is higher than atmospheric pressure, such as but not limited to about 1.1 atm, 1.5 atm, 2 atm, 2.5 atm, 3 atm, 3.5 atm, 4 atm, 4.5 atm, 5 atm, or any fractions or multiples thereof. In other embodiments, the reaction is run under reduced pressure, such as but not limited to about 0.1 atm, 0.2 atm, 0.4 atm, 0.5 atm, 0.6 atm, 0.8 atm, 0.9 atm or 0.95 atm.

In certain embodiments, the reaction is run using microwave irradiation.

The progress of the reaction may be monitored by methods known to those skilled in the art, such as but not limited to HPLC, silica or alumina chromatography, affinity chromatography, and the like.

Once the desired amount of product is obtained in the reaction mixture or the reaction is found to have reached equilibrium, the reaction may be quenched by removing one or more of the reagents (such as by evaporation, sublimation, precipitation, crystallization, solvent extraction, resin extraction, chromatography or the like), or by isolating the fructose product (such as by precipitation, crystallization, solvent extraction, resin extraction, chromatography or the like). Alternatively, the reaction mixture may be used as such, without significant manipulation or with simple centrifugation, decantation and/or filtration. The isolated fructose product may be further purified by a method known to one skilled in the art, such as but not limited to precipitation, crystallization, chromatography, solvent extraction, resin extraction or the like.

In certain embodiments, the reaction is quenched by adding an acidic reagent (such as but not limited to an acidic resin). In other embodiments, acidity introduced by virtue of adding an acidic reagent into the reaction mixture is partially or completed neutralized by adding a basic reagent (such as but not limited to a basic resin).

Compounds described herein may be purchased from commercial sources, synthesized from compounds available from commercial sources, or prepared using procedures described herein. In certain embodiments, hydroxyl groups on the glucose may be protected in order to avoid their unwanted participation in side reactions. Protecting groups are used to block some or all of the reactive moieties and prevent such groups from participating in chemical reactions until the protective group is removed. In certain embodiments, each protective group is removable by a different means. Protective groups that are cleaved under totally disparate reaction conditions fulfill the requirement of differential removal.

In certain embodiments, protective groups are removed by acid, reducing conditions (such as, for example, hydrogenolysis), and/or oxidative conditions. Groups such as trityl, dimethoxytrityl, acetal and t-butyldimethylsilyl are acid labile and used to protect hydroxy reactive moieties. In certain embodiments, hydroxy reactive moieties are blocked with protective groups removable by hydrogenolysis, such as the benzyl group. Allyl blocking groups are useful since they may be removed by metal or π-acid catalysts. Yet another form of protecting group is a resin to which a compound or intermediate is attached. As long as the residue is attached to the resin, that functional group is blocked and does not react. Once released from the resin, the functional group is available to react. Other protecting groups, plus a detailed description of techniques applicable to the creation of protecting groups and their removal are described in Greene & Wuts, Protective Groups in Organic Synthesis, 3^{rd} Ed., John Wiley & Sons, New York, N.Y., 1999, and Kocienski, Protective Groups, Thieme Verlag, New York, N.Y., 1994, which are incorporated herein by reference for such disclosure.

### Kits

The invention includes a kit comprising a calcium salt, organic base, applicator, and instructional material for use thereof. In certain embodiments, the instructional material included in the kit comprises instructions for isomerizing glucose into fructose. The instructional material recites the conditions under which the method of the invention should be implemented. In certain embodiments, the kit further comprises reagents or materials that can be used to purify and/or isolate fructose from the reaction mixture.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### Example 1

Glucose (180 mg) and calcium nitrate (240 mg) were added to a 5 mL dram vial with a stir bar. To this solution was added 0.5 mL D₂O and 0.5 mL trimethylamine (45% w/w in water). The reaction was stirred for 5 minutes, during which time the solids dissolved and the solution became slightly yellow. The solution was then transferred to an NMR tube and left at room temperature for 6 days. ¹³C NMR was used to analyze the reaction after incubation times of 1 hour, 1 day, 2 days, 3 days, and 6 days.

Initially the reaction mixture was primarily composed of a mixture of α-and β-glucopyranoside, as indicated by the following diagnostic peaks in the ¹³C NMR spectrum: (151 MHz, D₂O) δ100.27, 94.97, 78.17, 78.13, 77.95, 77.91, 77.79, 75.31, 74.26, 73.48, 72.25, 72.20, 71.98, 71.94, 63.11, 63.03. DSS was used as internal standard.

After 6 days, the α-and β-glucopyranosides were mostly consumed. The major product showed diagnostic peaks at the following resonances: ¹³C NMR (151 MHz, D₂O) δ111.72 C₂, 81.75 C₃, 78.78 C₄, 77.39 C₅, 68.41 (very broad) C₆, 60.20 C₁. DSS was used as internal standard. These peaks were indicative of a fructose calcium complex.

TABLE 1

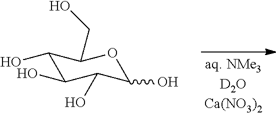 [SMILES: OCC1OC(O)C(O)C(O)C1O]

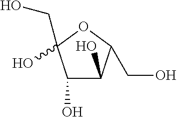

| Name | FW | Mole Equiv. | Mmoles | Amnt. | | Conc. | |
|---|---|---|---|---|---|---|---|
| glucose | 180 | 1 | 1 | 180 | mg | 1 | M |
| calcium nitrate | 164 | 1.5 | 1.5 | 240 | mg | 1.5 | M |
| trimethylamine | 59 | | | 0.5 | mL | | |
| (45% w/w in | | | | | | | |
| water) | | | | | | | |

### Example 2

Trimethylamine was added to a 100-mL round bottom flask. The system was cooled in ice bath, and glucose was added. Upon dissolution of glucose, calcium chloride was added, and the system was stirred while a homogeneous solution was formed. The system was warmed to room temperature. The reaction vessel was sealed with a rubber septum, and stirred at room temperature for 3 days.

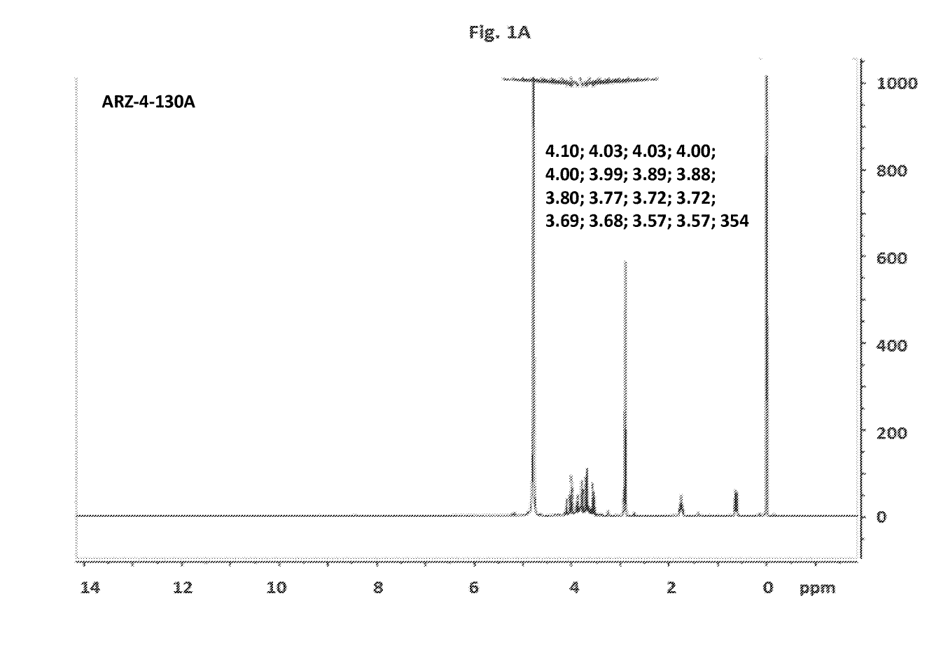

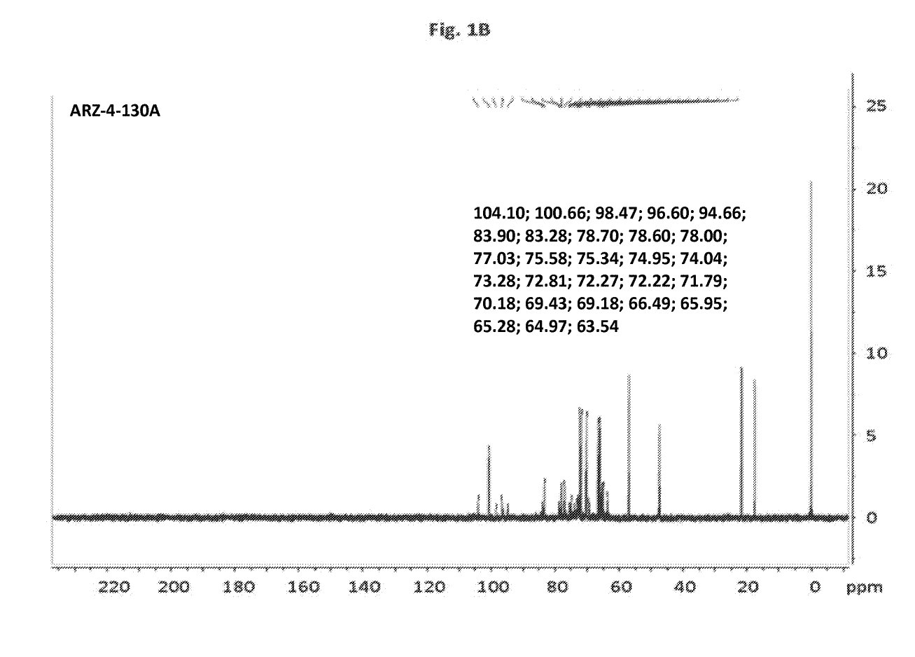

The reaction mixture was concentrated under reduced pressure in a rotoevaporator (15 minutes, 50° C., 10 mbar). An aliquot of the reaction was dissolved in D₂O, and analyzed by ¹H and ¹³C NMR spectroscopy. The spectra indicated that the reaction mixture comprised primarily fructose, with mannose and glucose as additional components (FIGS. 1A-1B).

The reaction mixture was diluted in 300 mL water, and 100 mL Dowex monosphere 850C resin (H⁺ form) were added. After 5 minutes, the reaction was acidic (pH ˜2) as indicated by pH paper. The solution was decanted, and 150 mL Dowex Monosphere 77 weak anion exchange resin (free base) were added. After 5 minutes, the reaction was mildly basic (pH ˜8) as indicated by pH paper. The system was decanted, and the supernatant was concentrated. The product was crystallized from ethanol/water, and placed in a refrigerator for 4 days. The product was filtered and the resulting crystals were placed under high vacuum overnight. 1.6 g of off-white crystals were isolated (with minor traces of contaminating trimethylamine). The isolated yield was 30%.

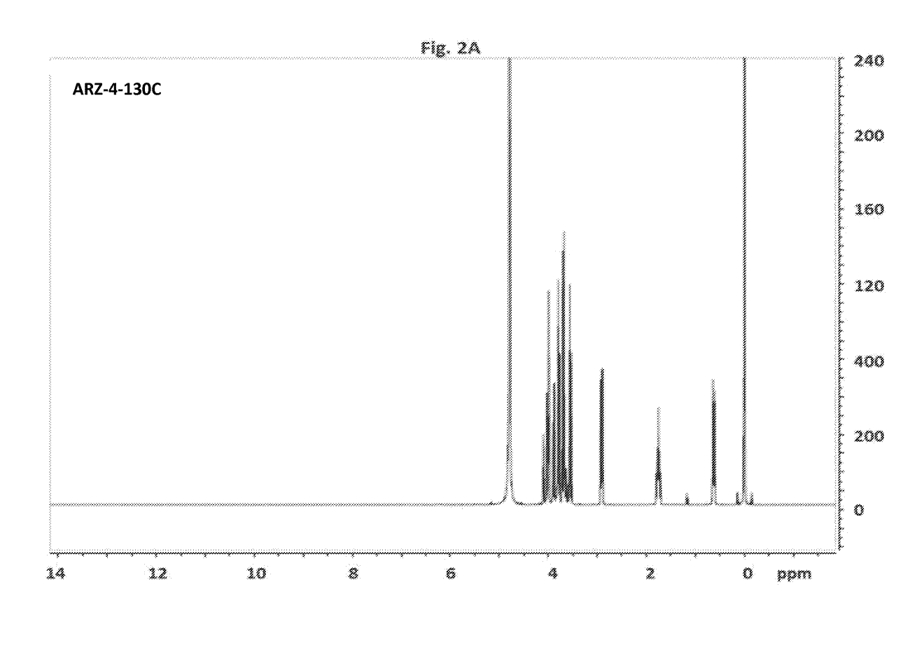

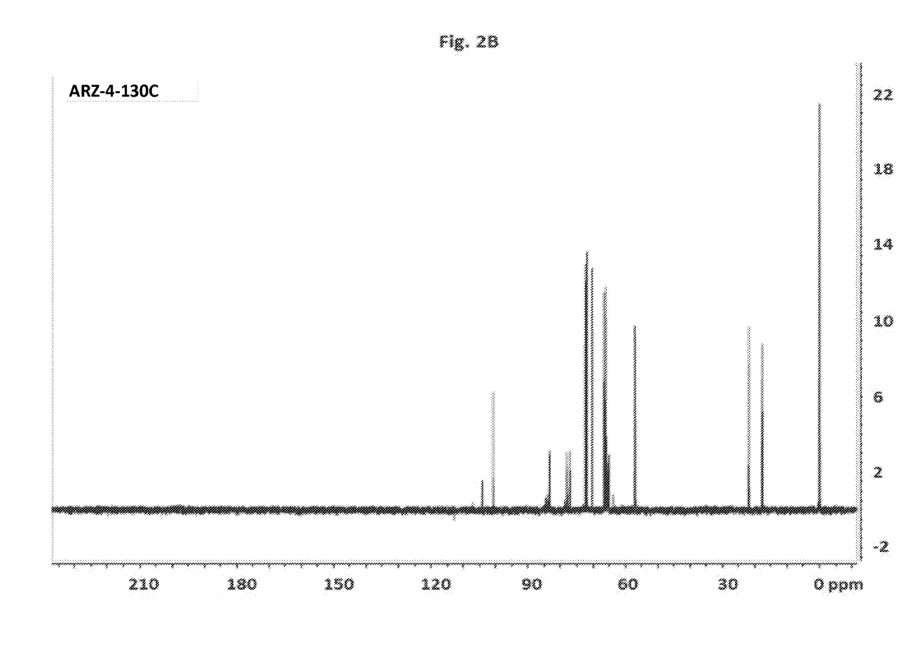

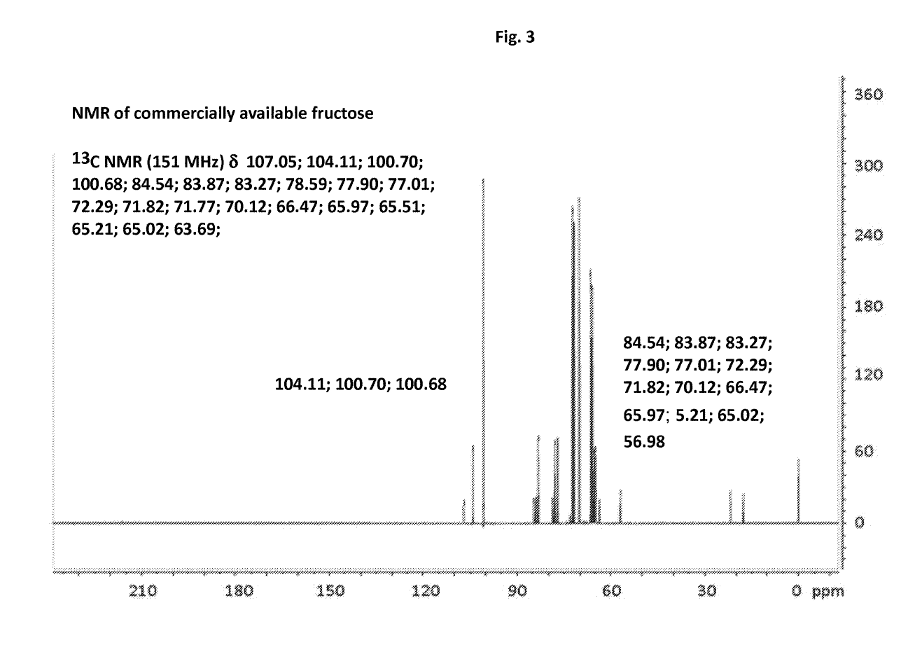

An aliquot of the product was dissolved in D₂O and analyzed by ¹H and ¹³C NMR spectroscopy (FIGS. 2A-2B). The isolated product was spectroscopically identical to commercially available fructose (FIG. 3).

TABLE 2

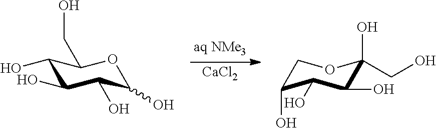

| | | Mole | | | |
|---|---|---|---|---|---|
| Name | FW | Equiv. | Mmoles | Amnt. | Conc. |
| glucose | 180 | 1 | 30 | 5.4 g | 1 M |
| calcium chloride | 111 | 1.5 | 45 | 5 g | 1.5 M |

### Example 3

The conversion of glucose to fructose was evaluated under various reaction conditions, and the evaluation included quantitation of the components in the equilibrium mixture and total recovery of sugars from the reaction mixture. The results are summarized in Table 3.

TABLE 3

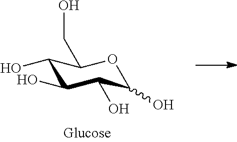

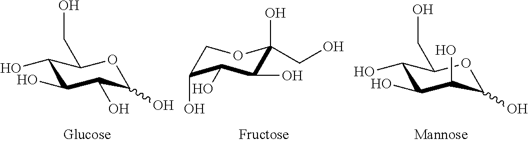

| | | | Equillibrium Mixture (%) | | | Total sugar |
|---|---|---|---|---|---|---|
| Catalyst | Temp. (° C.) | Time | Glucose | Mannose | Fructose | (% Recovery) |
| CaCl₂, NMe₃ | 25 | 1 day | 21 | 23 | 55 | 86.7 |
| CaCl₂, NMe₃ | 25 | 2 days | 8 | 15 | 77 | 72.6 |
| CaCl₂, NMe₃ | 25 | 3 days | 6 | 11 | 83 | 72.5 |
| none | 25 | 3 days | 99 | 0 | 1 | 102 |
| CaCl₂ | 25 | 3 days | 99 | 0 | 1 | 99.6 |
| NMe₃ | 25 | 3 days | 86 | 1 | 13 | 98.2 |
| CaCl₂, NMe₃ | 4 | 6 days | 24 | 20 | 56 | 83.4 |

As noted above and summarized in Table 4, the treatment of glucose with calcium chloride and trimethylamine at 25° C. afforded a yield of about 56% fructose after 2 days and about 60% fructose after 3 days.

TABLE 4

| | | % | % | % |
|---|---|---|---|---|
| | | Glu | Man | Fru |
| 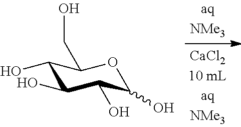 | 2 days 3 days | 5.9 4.5 | 10.7 8 | 56 60 |

The protocol used in the experiments illustrated in Table 4 is exemplified below.

| Name | FW | mol. eq. | mmol | amnt | Conc. |
|---|---|---|---|---|---|
| Glucose | 180 | 1 | 10 | 1.8 g | 1M |
| Calcium Chloride | 111 | 1.5 | 15 | 1.667 g | 1.5M |

Glucose and CaCl₂ were added to a 20 mL vial with stir bar. Trimethylamine was added and the system was stirred at room temperature.

After 20 minutes of reaction, 0.5 mL of the reaction were aliquotted, treated with 0.5 mL of D₂O and transferred to a NMR tube (sample: ARZ-4-187A).

After 24 hours of reaction, 0.5 mL of the reaction were aliquotted, treated with 0.5 mL of D₂O and transferred to a NMR tube (sample: ARZ-4-187B).

After 48 hours of reaction, 0.5 mL of the reaction were aliquotted, treated with 0.5 mL of D₂O and transferred to a NMR tube (sample: ARZ-4-187C).

The reaction mixture was diluted to 75 mL total volume with water. To the system was added 100 mL Dowex Monosphere 650 C Strong Cation exchange resin (H form). The system was filtered, and the resin was treated with 100 ml water for 5 minutes then filtered. The resin was washed again with 100 ml water.

All filtrates were combined in a solution, which was treated with 100 mL Amberlite IRA 67 weak anion exchange resin (free base). The resulting system was filtered, and the resin was treated with 100 mL water for . Allowed to soak for 5 minutes then filtered. The exchange resin was washed again with 100 mL water, and all filtrates were combined.

The final solution comprised 475 mL. A 10 ml aliquot was concentrated to dryness and dissolved in 1 mL water. This sample was analyzed on an Agilent 1100 HPLC fitted with a Biorad Aminex HPX87C calcium column and a Refractive Index Detector (0.6 mL/min water eluent, column at 80° C. and detector at 40° C.). Yield determined using a dn/dc value of 0.142.

Yield=(μg eluted)/(μL injected)*(total volume sample)/10/180 mg

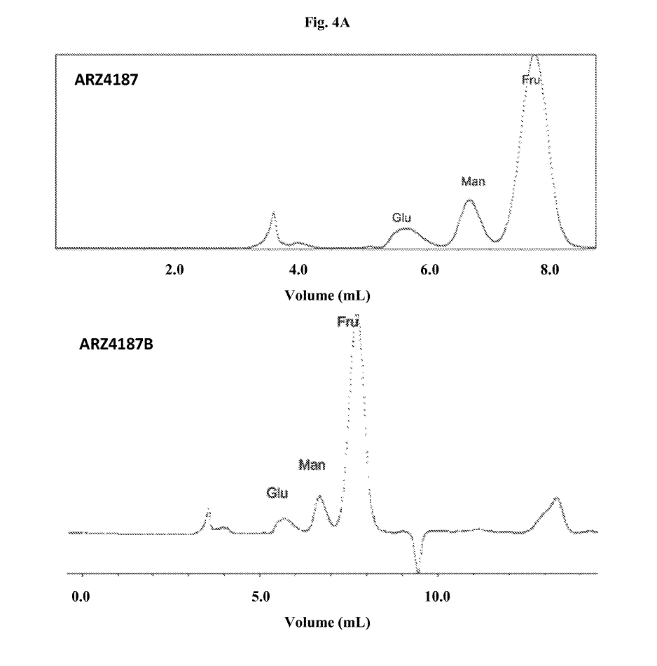

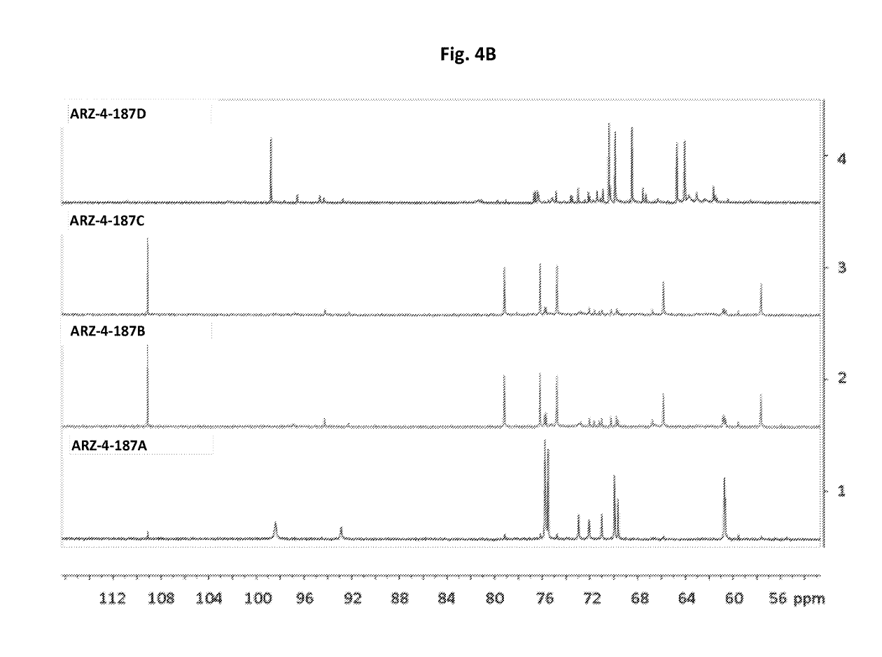

Experimental results are illustrated in FIGS. 4A-4B.

### Example 4

The methods of the reactions were exemplified using various sugar starting materials. The results are summarized in Table 5.

TABLE 5

| | Percentage of Sugar in Mixture |
|---|---|
| 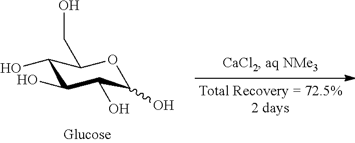 | 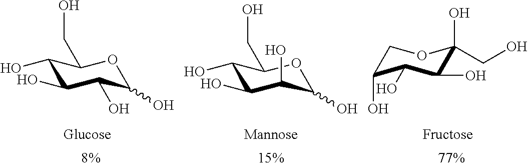 |
| 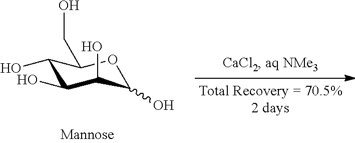 | 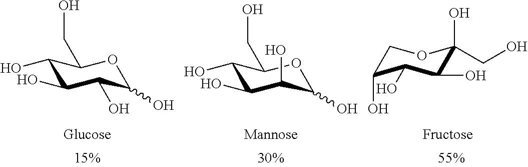 |
| 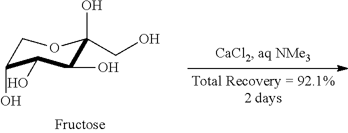 | 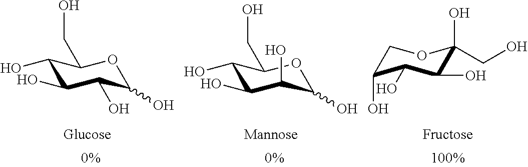 |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of isomerizing a sugar into fructose, wherein the sugar is selected from the group consisting of glucose and mannose, the method comprising contacting an aqueous solution of the sugar with a calcium salt and an organic base to form a system, wherein fructose is formed in the system.

2. The method of claim 1, wherein the system is free of organic solvents.

3. The method of claim 1, wherein the system comprises one or more water-soluble organic solvents.

4. The method of claim 1, wherein the calcium salt comprises one or more anions selected from the group consisting of chloride, bromide, iodide, nitrate, bicarbonate, sulfate bisulfate, phosphate, hydrogen phosphate, dihydrogen phosphate, and aliphatic, cycloaliphatic, aromatic, araliphatic, heterocyclic, carboxylic and sulfonic classes of organic anions.

5. The method of claim 1, wherein the organic base is aromatic or aliphatic.

6. The method of claim 5, wherein the aliphatic organic base is at least one selected from the group consisting of trimethylamine, triethylamine, tri-n-propylamine, diisopropylethylamine, N-alkyl-pyrrolidine, N-alkyl-morpholine, and N-alkyl-piperidine.

7. The method of claim 1, wherein the reaction is run from about 0° C. to about 100° C.

8. The method of claim 7, wherein the reaction is run from about 20° C. to about 50° C.

9. The method of claim 1, wherein the sugar, calcium salt and organic base are in solution.

10. The method of claim 1, wherein at least one selected from the group consisting of the calcium salt and the organic base is not in solution.

11. The method of claim 10, wherein at least one selected from the group consisting of the calcium salt and the organic base is a solid.

12. The method of claim 10, wherein at least one selected from the group consisting of the calcium salt and the organic base is immobilized on a solid support or permeable membrane.

13. The method of claim 10, wherein the calcium salt and the organic base are each independently immobilized on a solid support or permeable membrane.

14. The method of claim 1, wherein the reaction is run for a period ranging from about 5 minutes to about 4 weeks.

15. The method of claim 14, wherein the reaction is run for a period of about 3 days.

16. The method of claim 14, wherein the reaction is run for a period of about 2 weeks.

17. The method of claim 1, wherein the reaction is run under a pressure that is higher or lower than atmospheric pressure.

18. The method of claim 1, wherein the sugar, calcium salt and organic base are contacted in the presence of microwave irradiation.

19. The method of claim 1, wherein the reaction is quenched by a method comprising precipitation, filtration, decantation, crystallization, solvent extraction, resin extraction, resin neutralization or chromatography.

20. The method of claim 1, wherein the fructose is isolated from the system using a method comprising precipitation, filtration, decantation, crystallization, solvent extraction, resin extraction or chromatography.

21. The method of claim 1, wherein the system comprising fructose is further subjected to a process comprising precipitation, filtration, decantation, crystallization, solvent extraction, resin extraction or chromatography.
